(19) United States
(12) Patent Application Publication
(10) Pub. No.: US 2005/0203335 A1
(43) Pub. Date: Sep. 15, 2005

(54) Erection enhancement device

(76) Inventor: Joseph Stachon, Napa, CA (US)

Correspondence Address: Law Offices of Gerald L. Robertson, P.O. Box 5748, Napa, CA, 94581-0748, US

(21) Appl. No.: 10/751,293

(22) Filed: Dec. 31, 2003

(51) Int. Cl. A61F 5/00

(52) U.S. Cl. 600/39; 600/38

(57) ABSTRACT

A coil of tapered configuration fitted to the penis of a male human, said coil made from a malleable spring-like metal and coated with a substance which may be continually sanitized without adversely affecting said coil's physical properties. When placed on the penis said coil acts to establish and maintain engorgement of the penis with blood and provides a pumping action along the length of the penis which not only establishes, but maintains engorgement. The invention stimulates the penis enhancing feeling, stiffness, and also stimulates the contacting genitals of the female sexual partner.

### STATEMENT REGARDING FEDERALLY-SPONSORED RESEARCH AND DEVELOPMENT

The present invention has not been developed as a result of any federally funded project, contract or any other situation in which governmental involvement including money, employees, contractors or other support has existed or continues.

### FIELD OF THE INVENTION

The present invention deals with aids for sexual intercourse; specifically, an erection enhancement device.

### BACKGROUND OF THE INVENTION

Throughout time, there have been devices developed to enhance a male human's sexual performance; e.g. to assist in the maintenance of an erection during sexual intercourse. Medical evidence abounds to support the fact that the male erection is due from the physical perspective from the engorgement of the penis with blood, thereby stiffening and sensitizing the organ before and during intercourse.

Without addressing the complex emotional aspects of maintaining an erection, suffice it to say that an erection is enhanced when engorgement is prolonged and stimulus such as that from the pumping action associated with the act of intercourse is applied to the penis.

Various devices have been the subject of applications for patent and indeed, and for which protection has been granted. Among those most recent references are U.S. Pat. No. 6,015,379, granted to Sachse, for an erection aid, which discloses an elastic wrapping which essentially covers the penis and provides stiffening to assist in the sexual act.

Historically, one of the most relevant means to aid in maintaining an erection has been a ring placed at the base of the penis which helps reduce the amount of blood flowing back out of the penis, thereby prolonging erection.

In both of the above cases, one device either performs the job of the normally functioning penis—Sasche, and the other simply helps prolong engorgement—the ring. In neither case is there any function of the device to assist in stimulating the penis to assist in maintaining the erection.

Each of these references including the basic ring has an overriding deficiency in that they only address one aspect of maintenance of the erection, either maintaining engorgement of the penis with blood or providing a stiffening function in lieu thereof, but not the stimulus which is so beneficial in providing continuing arousal and establishing engorgement.

In U.S. Pat. No. 6,231,502, issued to McCarty, an erection is assisted by means of using a vacuum device with inflatable sliding seals to establish and maintain a vacuum, providing the operator time to install a retaining device, such as the ring mentioned hereinabove. In this instance, the establishment of an erection is assisted until a retaining device is then applied.

Another reference, U.S. Pat. No. 6,485,408, issued to Orten, comprises an elastic device adapted to be placed around at least a part of a penis, which device is in the form of a condom or a tightening ring. The device is equipped with a piezoelectric unit, for example a piezoelectric foil, for emission of stimulating vibrations. In such a case, the supply of current is via batteries.

The deficiencies in the above stem from several factors. In the Sasche patent, the device is wrapped almost entirely around the penis. Such a device can be a distraction in the act of intercourse. Likewise in the McCarty reference, time is required to establish the erection, which time is taken from the other acts of intimacy in which the participants might engage, thereby lessening the intimacy and providing a less enjoyable and stimulating experience.

What is needed is a device which not only helps maintain the erection by preventing loss of blood from the penis, like the aforementioned ring, but which also provides stimulus to the penis to assist in the maintenance of the engorgement. Of further aid would be a device which is easily removable, unlike the ring, which in some instances can be dangerously difficult to remove if the penis becomes too engorged.

Further, what is needed in the art is a way to lessen the time of distraction from the act of intercourse, thereby further stimulating the partners' excitement and enhancing the erection.

In addition, if such a device can be operated without any secondary form of energy, such as batteries or manual pumping action, then the simple act of intercourse may be less altered—a more natural experience may be had by the participants. How disappointing it would be to begin a moment of intimacy such as in the case of a man using the Orten device, only to find that the batteries are dead, and no replacements are at hand.

What is needed then, is a manual device which takes very little time to use, assists not only in maintaining engorgement of the penis, but also in stimulating the continual flow of blood thereto, and minimizes the “invasiveness” of the device, or the visual impact of the device on the participants.

If, on the other hand, the device also assisted the female during intercourse, in terms of excitement or arousal, and stimulation of the erogenous areas, that would be distinctive, as few, if any of the devices aside from Orten may assist in such regard, being primarily relegated to simply stiffening the penis or maintaining engorgement.

What is needed then, is an erection enhancing device which not only maintains engorgement, but stimulates blood flow, is easily applied and removed, and which also assists in the pleasure and stimulation of the female partner.

### SUMMARY OF THE INVENTION

It is an object of the present invention to provide an erection enhancement device which acts to maintain engorgement of the penis with blood in order to maintain an erection during sexual intercourse. Similar in some regard to the erection ring, applicant's invention employs a coil of malleable metal such as copper or aluminum, copper being the most desirable, such metal having been coated with polyvinyl chloride or plastic. At the base end, the end which would be that end adjacent to the male's abdomen during use, is a length of the coil which acts as a lever, allowing the diameter of the coiled device to be enlarged for easy application and removal. This is important to avoid an inability to remove the device as may happen in the case of an erection ring.

The device is tapered such that the base of the coil is slightly smaller in diameter than the top. Configured in this way, the constriction of the penis is achieved, not unlike the function of the basic erection ring, but unlike the ring, during use, a pumping action is achieved thereby not only maintaining engorgement of the penis with blood, but also stimulating the penis such that blood flow into the penis continues, thereby enhancing engorgement. This pumping action may be used to establish the erection to begin coitus, and naturally occurs during the act.

It is a further object of the present invention to provide stimulus to the female partner during intercourse. Applicant's invention also provides stimulus to the genitals of the female during the act of intercourse. The coil design, its size, spacing and pumping action also act in some ways like a ribbed condom, only more intense and not along the entire length of the penis, thereby giving the wearer enhanced pleasure as well as the female.

As a consequence of the structure of applicant's invention, the time required for a female to achieve orgasm is shortened, and the time for achievement of orgasm on the part of the male is lengthened, thereby approaching a synchronization of the partners' height of pleasure.

A tapering or sizing device is used in conjunction with the coiled aid to assist in the user sizing the device for personal use. In addition, because of the malleable nature of the invention, deformation may occur during removal of the device. The sizing device also functions to re-shape applicant's invention to its original conical shape for reuse. A simple tapered dowel-like structure having a taper of, for example, 4 degrees is sufficient to make the device of larger or smaller diameter.

### BRIEF DESCRIPTION OF THE DRAWINGS

FIG. 1 is a perspective view of the preferred embodiment showing the basic configuration of the invention.

FIG. 2 is a cross-sectional view of the preferred embodiment showing the core material and the applied coating in relative proportion.

FIG. 3 is a perspective of the sizing device used to shape applicant's invention into the desired inside diameter.

### DETAILED DESCRIPTION OF THE INVENTION

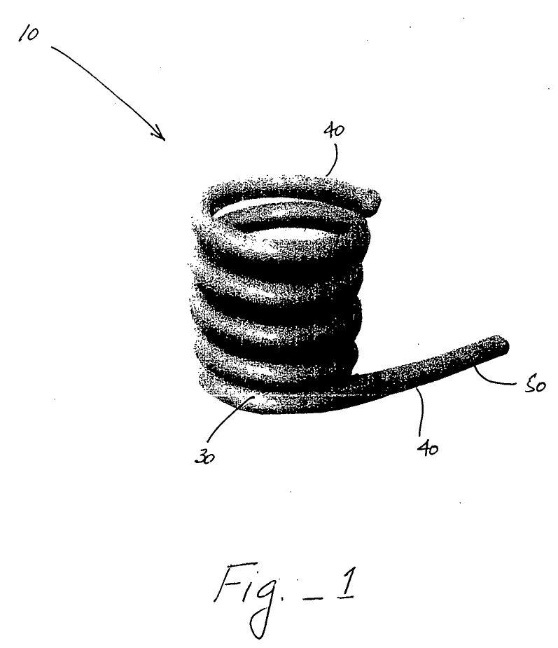

As seen in FIG. 1, applicant's device 10, comprises a tapered coil of spring-like material. Coil 10 is tapered for two reasons: first, to constrict the base of the penis to maintain engorgement of the penis with blood; and second, to assist in the aforementioned pumping action of the device in assisting in establishing an erection and during use. A core 20 is surrounded by a layer 30 of a soft material having preferred frictional characteristics.

At each end 40 of the device, coil is angled slightly off axis and rounded to relieve pressure against the penis when engaged, thereby increasing comfort. A length of the device at the base end is slightly elongated to form a lever 50, which is used to temporarily increase the diameter of the device for application and removal.

In the preferred embodiment, the malleable metal used is 14 guage single strand copper wire, coated with polyvinyl chloride (“PVC”) in a thickness of approximately 0.18″. While PVC is the preferred coating, any suitable plastic or other material which is easy to sanitize and disinfect, and is essentially non-porous may be employed.

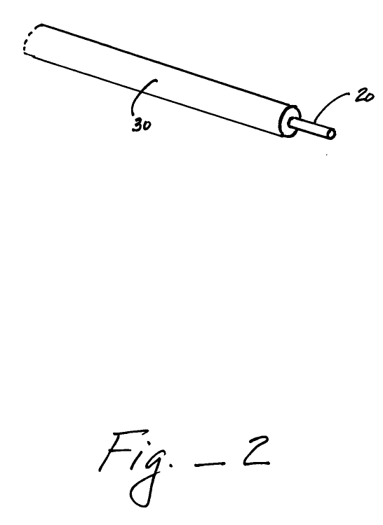

FIG. 2 shows a cross-section of the coil material and the relative thickness of the PVC coating on the malleable metal core, 20.

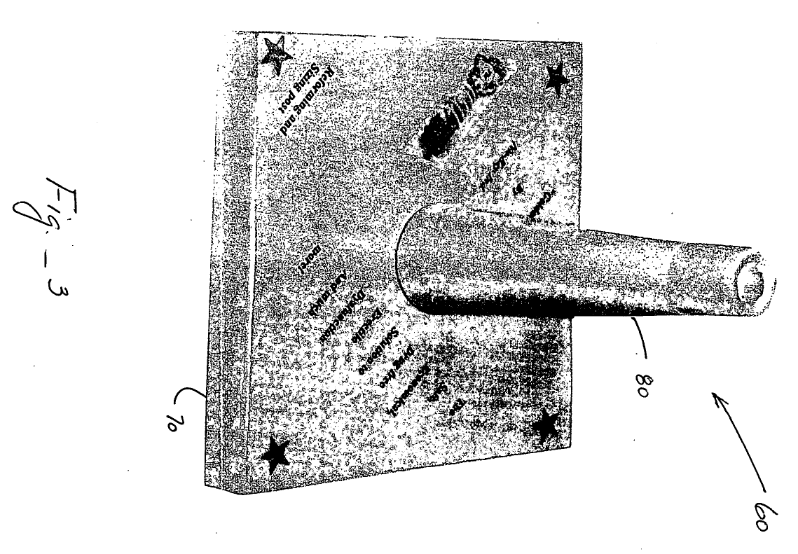

FIG. 3 shows the sizing device 60, comprising a base 70 and sizer 80. Sizer 80 is simply a tapered dowel of wood or plastic which is tapered approximately 4 degrees. Sizer 80 is preferably releasably mounted into a receptacle in base 70, said receptacle being threaded in the preferred embodiment to accept a male threaded structure at the mating end of sizer 80. Coil 10 is slid upside down over sizer 80 to the desired diameter, and the ends tensioned to establish the desired inside diameter. Any malleable metal may be employed as core 20 if it may be so shaped. In sizing, the space between coil strands should be preferably approximately 1/8″ to ¼″, depending on the size of the user, to reduce the risk of pinching the skin of the penis. This spacing acts to provide the gentle but effective pumping action during intercourse.

At any time during the act, or preferably after successful coitus, lever 50 may be used to gently expand coil 10 for removal.

While the invention has been described in connection with what is presently considered the most practical and preferred embodiment(s), it is to be understood that the invention is not limited to the disclosed embodiment(s) but, on the contrary is intended to cover various modifications and equivalent arrangements included within the scope of the appended claims.

1. An erection enhancement device comprising:
a malleable core material wound in a tapered coil, the ends of said coil slightly bent away from the circular axis of said coil, said end of said coil located proximate to the smallest diameter of said coil being elongated forming a lever, said lever acting to increase the inside diameter of said coil when pressed; and
a coating applied to said core material, said coating being easily cleaned or sanitized.

2. The device of claim 1 wherein:
said core material is comprised of single strand copper.

3. The device of claim 1 wherein:
said coating is comprised of polyvinyl chloride.

4. The device of claim 1 further comprising:
a base having a receptacle therein;
an elongated, substantially conical sizer mounted in said receptacle normal to said base for establishing a desired inside diameter of said coil.
